(19) United States
(12) Patent Application Publication
(10) Pub. No.: US 2025/0127968 A1
(43) Pub. Date: Apr. 24, 2025

(54) BREAST PUMPS

(71) Applicant: Mother's Milk dba Spectra Baby USA, Davie, FL (US)

(72) Inventors: Samuel Bernard Levey, Coral Springs, FL (US); Heidi Perlman-Humphries, Southwest Ranches, FL (US)

(21) Appl. No.: 18/493,269

(22) Filed: Oct. 24, 2023

(51) Int. Cl. A61M 1/06

(52) U.S. Cl. CPC A61M 1/0693; A61M 2205/581; A61M 2205/587

(57) ABSTRACT

A breast pump including a vacuum source, a breast engagement portion, and a controller. The breast engagement portion is in fluid communication with the vacuum source and is configured to engage a breast of a user to express breast milk. The controller is in electrical communication with the vacuum source and is configured to notify a user that one or more components of the breast engagement portion require maintenance or replacement.

### TECHNICAL FIELD

The present invention relates generally to breast pumps and, more specifically, to breast pumps that are configured to notify a user when one or more components require maintenance or replacement.

### BACKGROUND

Breast pumps are typically used by lactating mothers to collect breast milk for feeding an infant. For ease of use, efficacy, and efficiency reasons, powered electric breast pumps have become standard. Powered electric breast pumps can include various components (e.g., flanges, valves, backflow protectors, tubing, vacuum source(s), containers, etc.) that allow for the breast pumps to express milk from a user's breast.

### SUMMARY

An example breast pump includes a vacuum source, a breast engagement portion, and a controller. The breast engagement portion is in fluid communication with the vacuum source and is configured to engage a breast of a user to express breast milk. The controller is in electrical communication with the vacuum source and is configured to notify a user that one or more components of the breast engagement portion require maintenance or replacement.

Another example breast pump includes a vacuum source, a breast engagement portion, and a controller. The breast engagement portion is in fluid communication with the vacuum source and is configured to engage a breast of a user to express breast milk. The controller is in electrical communication with the vacuum source and is configured to calculate a historical average pumping session time for the user, a statistically significant variation of the historical average pumping session time, and a recent average pumping session time for the user based on a determination that the vacuum source is in an activated configuration. The controller is further configured to notify a user that one or more components of the breast engagement portion require maintenance or replacement when the recent average pumping session time is greater than the statistically significant variation of the historical average pumping session time.

Another example breast pump includes a vacuum source, a breast engagement portion, and a controller. The breast engagement portion is in fluid communication with the vacuum source and is configured to engage a breast of a user to express breast milk. The controller is in electrical communication with the vacuum source and is configured to calculate a cumulative operating time of the breast pump based on a determination that the vacuum source is in an activated configuration. The controller is further configured to notify a user that one or more components of the breast engagement portion require maintenance or replacement when the cumulative operating time exceeds a predetermined value.

An example controller for a breast pump is configured to calculate a cumulative operating time of the breast pump, a recent average pumping session time for a user, a historical average pumping session time, and a statistically significant variation of the historical average pumping session time. The controller is further configured to determine that at least one of the cumulative operating time exceeds a predetermined cumulative operating time value and the recent average pumping session time exceeds the statistically significant variation of the historical average pumping session time. The controller is further configured to activate a notification element to inform a user that one or more components of the breast pump require maintenance or replacement when the controller determines that at least one of the cumulative operating time exceeds the predetermined cumulative operating time value and the recent average pumping session time exceeds the statistically significant variation of the historical average pumping session time.

Another example controller for a breast pump is configured to calculate a cumulative operating time of the breast pump, a recent average pumping session time for a user, a historical average pumping session time, and a statistically significant variation of the historical average pumping session time. The controller is further configured to determine that both the cumulative operating time exceeds a predetermined cumulative operating time value and the recent average pumping session time exceeds a statistically significant variation of the historical average pumping session time. The controller is further configured to activate a notification element to inform a user that one or more components of the breast pump require maintenance or replacement when the controller determines that both the cumulative operating time exceeds the predetermined cumulative operating time value and the recent average pumping session time exceeds the statistically significant variation of the historical average pumping session time.

### BRIEF DESCRIPTION OF THE DRAWINGS

FIG. 1 is a schematic view of an example breast pump;

FIG. 2 illustrates another example breast pump;

FIG. 3 illustrates an exploded view of a breast engagement portion for the breast pump of FIG. 2;

FIG. 4 illustrates an example method performed by a controller of a breast pump; and

FIG. 5 illustrates another example method performed by a controller of a breast pump.

### DETAILED DESCRIPTION

The Detailed Description describes exemplary embodiments of the invention and is not intended to limit the scope of the claims in any way. Indeed, the invention is broader than and unlimited by the exemplary embodiments, and the terms used in the claims have their full ordinary meaning. Features and components of one exemplary embodiment may be incorporated into the other exemplary embodiments. Inventions within the scope of this application may include additional features, or may have less features, than those shown in, and described in connection with, the exemplary embodiments.

As described herein, the terms “substantially” and “about” are defined as at least close to (and includes) a given value or state (preferably within 10% of, more preferably within 1% of, and most preferably within 0.1% of a given value or state).

Powered electric breast pumps can include various components (e.g., flanges, valves, backflow protectors, tubing, vacuum source(s), containers, etc.) that allow for the breast pumps to express breast milk from a user's breast. Some of these components may require maintenance or replacement to maintain optimum performance over time. For example, breast pumps can include a diaphragm assembly and/or a one-way valve that affect suction within a breast engagement portion of the breast pump during use of the breast pump. Damage or wear and tear to these components can decrease the suction within the breast engagement portion and, consequently, decrease the performance of the breast pump. Such decreased performance can lead to lower efficacy, increased pumping times, and potentially the inability of a user to maintain a milk supply when using the breast pump.

The breast pumps described herein are configured to notify a user as to when one or more components of the breast pump require maintenance or replacement. For example, the breast pumps described herein include a controller that is configured to notify a user when maintenance or replacement is required. Notification to a user when one or more components of a breast pump require maintenance or replacement is advantageous because most users are unaware that such maintenance or replacement is required, which leads to the decreased performance of the breast pump and, consequently, a decreased milk supply. Sufficient maintenance and/or replacement of breast pump components can allow for ensuring milk supply of a user, enabling a user to manage pumping time to prevent extensions caused by decreased performance, enabling a user to schedule pumping times within available time periods, and a reduction in service calls and troubleshooting within company manufacturing and/or selling the breast pumps. Such a notification is also advantageous because it could allow a user to become aware of potential problems that may require medical attention. For example, if the breast pump is indicating that maintenance or replacement is required, but the user has already performed such maintenance or replacement, then the notification could inform the user that the decreased milk supply may relate to a medical condition (e.g., an infection) that requires attention from a medical professional. Accordingly, the breast pumps described herein allow for a user to easily determine if there is a problem with one or more components of the breast pump, as well as inform a user that the user potentially has a health condition that requires medical attention.

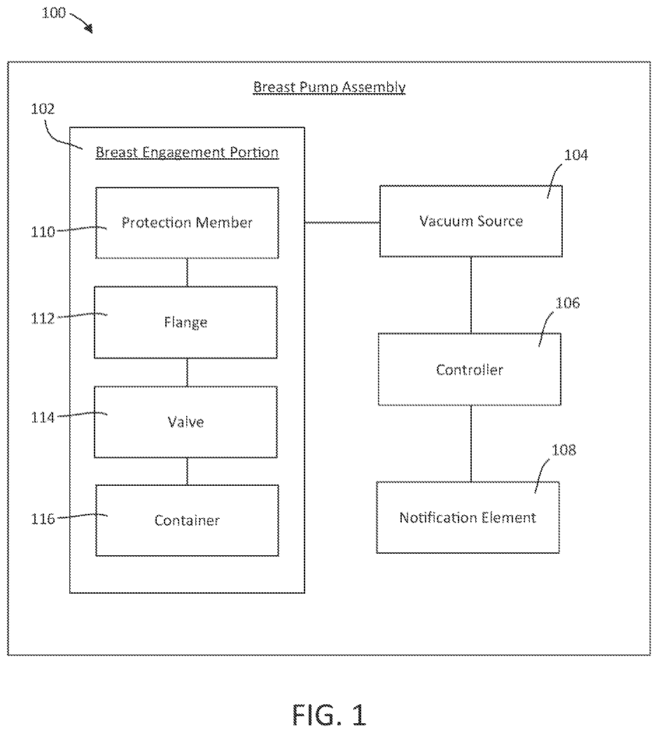

FIG. 1 illustrates an example of breast pump 100 that includes a breast engagement portion 102, a vacuum source 104, a controller 106, and a notification element 108. The vacuum source 104 can be fluidly connected to the breast engagement portion 102 such that activation of the vacuum source 104 causes the breast engagement portion 102 to engage a user's breast to express milk therefrom. For example, the vacuum source 104 can be configured to provide a cyclical vacuum pressure to the breast engagement portion 102 that causes the breast engagement portion 102 to engage the user's breast in a manner that emulates an infant engaging the user's breast to express milk therefrom.

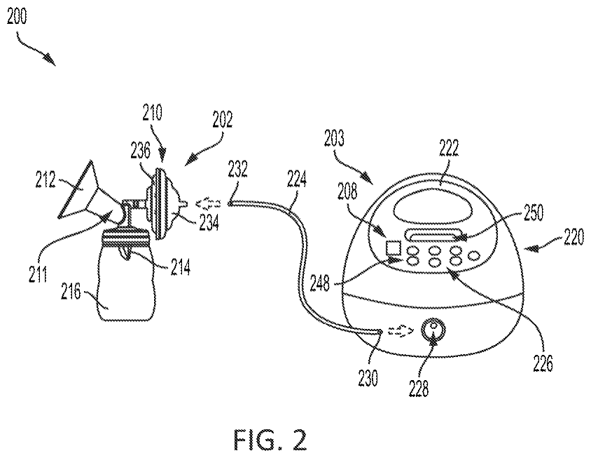

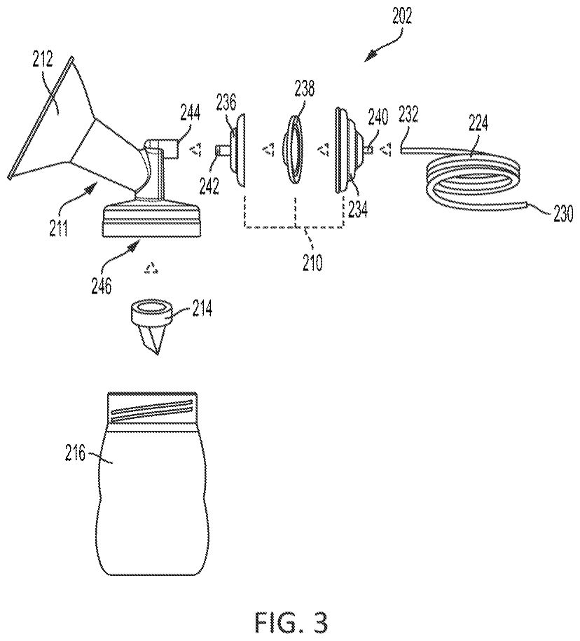

In some examples, the breast engagement portion 102 includes a protection member 110, a flange 112, a one-way valve 114, and a container 116. Activation of the vacuum source 104 causes suction within the flange 112 such that the flange 112 engages the user's breast to express milk therefrom. The protection member 110 provides a layer of protection between the flange 112 and the vacuum source 104 such that milk being expressed from the user's breast is prevented from moving into the vacuum source 104. In some instances, the protection member 110 can include diaphragm assembly (e.g., the diaphragm assembly 210 shown in FIGS. 2-3), where the vacuum source 104 causes movement of the diaphragm assembly to create suction in the flange 112. However, the protection member 110 can take any suitable form that allows for the vacuum source to cause suction within the flange 112 while also preventing milk from moving from the flange 112 and into the vacuum source 104. After the milk is expressed from a user's breast, the milk then moves through the one-way valve 114 and into the container 116. The one-way valve 114 can be configured to allow for an increase in suction levels within the flange 112, which helps realize the performance intent of the breast pump 100. The one-way valve 114 can be, for example, a duckbill valve or any other suitable valve that is capable of allowing the pump to create, hold, and maintain the desired suction within a flange while also preventing backflow of breast milk from the container 116 and into the flange 112. The container 116 can take any suitable form that is capable of receiving and holding breast milk. For example, the container 116 can be a rigid container, flexible bag, or take any other suitable form. In various examples, the vacuum source 104 can be connected to the breast engagement portion 102 via one or more tubes (e.g., the tubing 224 shown in FIGS. 2-3).

The controller 106 is configured to cause the vacuum source 104 to move between activated and deactivated configurations (e.g., via instructions from a user). For example, a user can engage a user interface (e.g., the user interface 226 shown in FIG. 2) to turn the breast pump 100 to an on configuration and cause the controller 106 to activate the vacuum source 104, and a user can engage the user interface to turn the breast pump 100 to an off configuration and cause the controller 106 to deactivate the vacuum source 104. In some instances, the user can select a setting for a desired amount of time for a pumping session and/or a desired vacuum pressure for a pumping session. The controller 106 can take any suitable form that is capable of controlling operation of the breast pump 100. The breast pump 100 can include a power source (not shown) for providing power to the vacuum source 104 and the controller 106. For example, the breast pump 100 can be battery powered or wired into an electrical system.

The controller 106 can be configured to determine when one or more components of the breast pump 100 require maintenance or replacement. The controller 106 can include processing circuitry that is configured to monitor the operation of the breast pump (e.g., monitor when the vacuum source 104 is activated) and calculate values relating to the use of the breast pump, such as, for example, cumulative time in which the vacuum source 104 is activated, time of an individual pumping session, average time of pumping sessions, historical average time of pumping sessions, a statistically significant variation of the historical average time of pumping sessions, a recent average time of pumping sessions, or any other time relating to the use of the breast pump 100. The controller 106 can also be configured to compare one or more of these calculated times to predetermined values and notify a user based on the comparison between the calculated times and predetermined values. For example, if the cumulative time in which the vacuum source is activated exceeds a predetermined value (e.g., an amount of time recommended by a manufacturer for replacement of one or more components based on design criteria of the components), the controller 106 can be configured to send a signal to a notification element that informs the user that one or more components of the breast pump require maintenance or replacement. In various examples, if a recent average time of pumping sessions exceeds a predetermined value (e.g., a statistically significant variation of a historical average time of pumping sessions), then the controller 106 can be configured to send a signal to a notification element that informs the user that one or more components of the breast pump require maintenance or replacement.

In some examples, the controller 106 can be configured to adjust to a specific user. For example, the controller can track a time for each pumping session by a user, and this tracked information can be used to adjust the predetermined value that is used to determine if any maintenance or replacement of components is required. In various examples, the tracked pumping session times can be used to create a historical average pumping session time, a statistically significant variation of the historical average pumping session time, and a recent average pumping session time. In these examples, the controller 106 can be configured to compare the recent average pumping session time to the statistically significant variation of the historical average pumping session time to determine if any maintenance or replacement of components is required. For example, if the recent average pumping session time exceeds the statistically significant variation of the historical average pumping session time, then the controller 106 determines that maintenance or replacement of component(s) is required. In some examples, the most recent single pumping session time (rather than the recent average pumping session time) can be compared to the statistically significant variation of the historical average pumping session time to determine if any maintenance or replacement of components is required.

The recent average pumping session time can be, for example, the average time for the previous two most recent pumping sessions, three most recent pumping sessions, four most recent pumping sessions, five more recent pumping sessions, etc. The historical average pumping session time can be the average pumping session for every pumping session by the user less the number of recent pumping session times used to calculate the recent average pumping session time. Alternatively, the historical average pumping session time can be the average time from predetermined pumping sessions (e.g., the first pumping session through the twentieth pumping session, the fifth pumping sessions through the fifteenth pumping session, or the like). The statistically significant variation of the historical average pumping session time can be a predetermined percentage greater than the historical average pumping session time, such as, for example, between about 2% and about 50% greater than the historical average pumping session time, such as between about 5% and about 50% greater, such as between about 5% and about 25% greater, such as between about 5% and about 20% greater, such as between about 10% and about 15% greater. Alternatively, the statistically significant variation of the historical average pumping session time can be a predetermined time value greater than the historical average pumping session time, such as, for example, between 2 minutes and 20 minutes greater than the historical average pumping session time, such as between about 2 minutes and about 15 minutes greater, such as between about 2 minutes and about 10 minutes greater, such as between about 2.5 minutes and about 7.5 minutes greater.

The notification element 108 can take any suitable form that allows for the controller 106 to inform a user that one or more components of the breast pump 100 require maintenance or replacement. For example, the notification element 108 can be an audible alert, a visual alert displayed on a housing (not shown) of the pump assembly 100, an alert sent to an external device (e.g., a phone, a tablet, a computer), or any combination thereof. The visual alert can include an alert on a display screen (e.g., display screen 250 shown in FIG. 2), such as a symbol, text, backlighting on the display (e.g., a red background light or other atypical color), or the like. A housing of the device can include a light or button or the like that is configured illuminate when the notification element 108 is activated. In examples that include sending an alert to an external device, the controller 106 can be configured to wirelessly transmit the notification alert to the external device (e.g., via Bluetooth, Wi-Fi, etc.). For example, the alert can be sent to the external device by a software application notification, an email, a text message, or by any other suitable means. In these examples, the notification could also include a link to replacement component(s) that allows for a user to easily purchase such component(s).

The controller 106 can be configured to inform a user that a specific component needs maintenance or replacement, or the controller 106 can be configured to send a general alert to the user without specifying a specific component. For example, the notification element 108 can include multiple indicators with each component of the breast pump that may require maintenance or replacement having an indicator associated therewith. In some examples, text on a display can indicate specific component(s) that require maintenance or replacement, or an alert to an external device can specify which component(s) require maintenance or replacement.

In some instances, the breast pump 100 can include one or more sensors (not shown) that are configured to send signals to the controller 106 to cause the controller to activate the notification element 108 and inform a user that one or more components of the breast pump 100 require maintenance or replacement. The sensor(s) can be positioned at any desired location on the breast pump and be configured to send a signal to the controller 106 when the sensor(s) detect information that can be used by the controller 106 to determine that one or more components require maintenance or replacement. The sensor(s) can include, for example, flow sensors, pressure sensors, proximity sensors, a sensor capable of detecting volume of liquid in a container, or any combination thereof. However, sensors can be costly and cause the breast pump 100 to be unnecessarily complex. Accordingly, while some implementations can include sensors, it should be understood that various implementations of the breast pumps are configured such that the controller 106 can determine whether one or more components of the breast pump require maintenance or replacement without obtaining information from sensor(s) to do so.

Referring to FIG. 1, the breast pump 100 can take any suitable form for an electrically powered breast pump. For example, the breast pump 100 can be a portable or non-portable pump that includes a housing (not shown) for holding the vacuum source 104 and controller 106, and the breast engagement portion 102 can be configured to attached to the housing via one or more tubes. In these embodiments, the housing can include a notification element 108 that notifies a user of that one or more components of the breast pump 100 require maintenance or replacement. In other examples, the breast pump 100 can be a “hands-free” design in which the breast pump 100 (including the vacuum source 104 and the controller 106) is configured to fit within a bra of the user. In these examples, the breast pump 100 may include a housing (not shown) that fits within the bra of the user and the housing can include a notification element 108 that notifies a user of that one or more components of the breast pump 100 require maintenance or replacement. In some instances, the controller 106 can be included in a device that is configured to be removably electrically connected to a breast engagement portion and/or vacuum source of a breast pump such that the controller 106 can calculate values relating to the use of the breast pump and determine when one or more components of the breast pump 100 require maintenance or replacement. In any of these examples, the one or more components that require maintenance or replacement can be components of the breast engagement portion 102 (e.g., tubing, the protection member 110, the flange 112, the one-way valve 114, and/or the container 116) or other components of the breast pump 100 (e.g., the vacuum source 104).

While the breast pump 100 is described as having a breast engagement portion 102 having a protection member 110, a flange 112, a one-way valve 114, and a container 116, it should be understood that the breast engagement portion 102 can take any other suitable form that is capable of engaging a breast of a user to express milk therefrom. Accordingly, the breast engagement portion 102 can have any additional or alternative components and the controller 106 can be configured to notify a user when one or more of these components require maintenance or replacement.

FIGS. 2-3 illustrate another example of a breast pump 200 that includes a breast engagement portion 202 and a vacuum and control portion 203. The vacuum and control portion 203 can include a housing 220, a vacuum source (not shown) disposed within the housing 220, a controller (not shown) disposed within the housing 220, and a notification element 208. In the illustrated example, the housing 220 includes a handle 222 for engagement by a user, which allows for the breast pump 200 to be portable. In other examples, the housing 220 may not be portable such that a user must remain proximate the vacuum and control portion 203 when using the breast pump 200.

The breast engagement portion 202 can include tubing 224, a diaphragm assembly 210, a breast shield 211, a one-way valve 214, and a container 216. A first end 230 of the tubing 224 can be connected to the housing 220 of the vacuum and control portion 203 via a port 228 such that the tubing is in fluid communication with the vacuum source, and a second end 232 of the tubing 224 can be connected to the diaphragm assembly 210 such that the vacuum source can be fluidly connected to the diaphragm assembly 210. In the illustrated example, the diaphragm assembly 210 has a first diaphragm housing portion 234, a second diaphragm housing portion 236, and a flexible membrane 238 positioned within the first and second diaphragm housing portions 234, 236. The first diaphragm housing portion 234 includes an opening 240 for fluidly connecting to the second end 232 of the tubing 224, and the second diaphragm housing portion 236 has an opening 242 for fluidly connecting to the breast shield 211 via a breast shield opening 244. The breast shield 211 has a flange 212 that is configured to engage a user's breast and is in fluid communication with the breast shield opening 244, and the breast shield 211 has a main opening 246 that is in fluid communication with the container 216. The one-way valve 214 is positioned between the main opening 246 of the breast shield 211 and the container 216.

Activation of the vacuum source of the vacuum and control portion 203 causes a cyclical suction force within the tubing 224 and, consequently, the opening 240 of the diaphragm assembly 210 such that the cyclical suction force is applied to the flexible membrane 238. This force causes the flexible membrane 238 to move within the diaphragm housing 234, 236 and create a cyclical suction force within the flange 212 of the breast shield 211 via the fluid connection between the second diaphragm housing portion 236 and the breast shield opening 244. This cyclical suction force cause the flange 212 to engage the breast of a user to express milk therefrom. That is, the cyclical suction force is configured to provide a cyclical vacuum pressure within the flange 212 that causes the flange 212 to engage the user's breast in a manner that emulates an infant engaging the user's breast to express milk therefrom. The one-way valve 214 is configured to allow for an increase in suction levels within the flange 212, which helps realize the performance intent of the breast pump 200. The milk expressed from the user's breast moves from the flange 212, through the main opening 246 of the breast shield 211, through the one-way valve 214, and into the container 216. In the illustrated example the one-way valve 314 is a duckbill valve. However, the one-way valve 214 can take any other suitable form that is capable of allowing the pump to create, hold, and maintain the desired suction within a flange 212 while also preventing backflow of breast milk from the container 216 and into the breast shield 211. In the illustrated example, the container 216 is a rigid container. However, the container 216 can take any other suitable form that is capable of receiving and holding breast milk.

The controller (not shown), which is disposed within the housing 220, is configured to cause the vacuum source to move between activated and deactivated configurations (e.g., via instructions from a user). For example, a user can engage a user interface 226 to turn the breast pump 200 to an on configuration and cause the controller to activate the vacuum source, and a user can engage the user interface 226 to turn the breast pump 200 to an off configuration and cause the controller to deactivate the vacuum source. The user interface 226 can include one or more buttons 248 that allow for a user to select desired settings form the breast pump 200. For example, a user can select a desired amount of time for a pumping session and/or a desired vacuum pressure for a pumping session. The user interface 226 can include a display screen 250 for conveying information to a user (e.g., session time, pressure, etc.). The controller can take any suitable form that is capable of controlling operation of the breast pump 200. The breast pump 200 can include a power source (not shown) for providing power to the vacuum source and the controller. For example, the breast pump 200 can be battery powered or wired into an electrical system.

The controller can be configured to determine when one or more components of the breast pump 200 require maintenance or replacement. In certain situations, the diaphragm assembly 210 and/or one-way valve 212 may be worn or otherwise defected, which affects the performance of the breast pump 200. In these situations, the controller can notify a user (e.g., via notification element 208) that the diaphragm assembly 210 and/or the one-way valve 212 (or any other component of the breast pump 200) require maintenance or replacement.

The controller can include processing circuitry that is configured to monitor the operation of the breast pump (e.g., monitor when the vacuum source is activated) and calculate values relating to the use of the breast pump 200, such as, for example, cumulative time in which the vacuum source is activated, time of an individual pumping session, average time of pumping sessions, historical average time of pumping sessions, a statistically significant variation of the historical average time of pumping sessions, a recent average time of pumping sessions, or any other time relating to the use of the breast pump 200. The controller can also be configured to compare one or more of these calculated times to predetermined values and notify a user based on the comparison between the calculated times and predetermined values. For example, if the cumulative time in which the vacuum source is activated exceeds a predetermined value (e.g., an amount of time recommended by a manufacturer for replacement of one or more components based on design criteria of the components), the controller can be configured to send a signal to a notification element 208 that informs the user that one or more components of the breast pump require maintenance or replacement. In various examples, if a recent average time of pumping sessions exceeds a predetermined value (e.g., a statistically significant variation of a historical average time of pumping sessions), then the controller can be configured to send a signal to a notification element 208 that informs the user that one or more components of the breast pump require maintenance or replacement.

In some examples, the controller can be configured to adjust to a specific user. For example, the controller can track a time for each pumping session by a user, and this tracked information can be used to adjust the predetermined value that is used to determine if any maintenance or replacement of components is required. In various examples, the tracked pumping session times can be used to create a historical average pumping session time, a statistically significant variation of the historical average pumping session time, and a recent average pumping session time. In these examples, the controller can be configured to compare the recent average pumping session time to the statistically significant variation of the historical average pumping session time to determine if any maintenance or replacement of components is required. For example, if the recent average pumping session time exceeds the statistically significant variation of the historical average pumping session time, then the controller determines that maintenance or replacement of component(s) is required. In some examples, the most recent single pumping session time (rather than the recent average pumping session time) can be compared to the statistically significant variation of the historical average pumping session time to determine if any maintenance or replacement of components is required.

The recent average pumping session time can be, for example, the average time for the previous two most recent pumping sessions, three most recent pumping sessions, four most recent pumping sessions, five more recent pumping sessions, etc. The historical average pumping session time can be the average pumping session for every pumping session by the user less the number of recent pumping session times used to calculate the recent average pumping session time. Alternatively, the historical average pumping session time can be the average time from predetermined pumping sessions (e.g., the first pumping session through the twentieth pumping session, the fifth pumping sessions through the fifteenth pumping session, or the like). The statistically significant variation of the historical average pumping session time can be a predetermined percentage greater than the historical average pumping session time, such as, for example, between about 2% and about 50% greater than the historical average pumping session time, such as between about 5% and about 50% greater, such as between about 5% and about 25% greater, such as between about 5% and about 20% greater, such as between about 10% and about 15% greater. Alternatively, the statistically significant variation of the historical average pumping session time can be a predetermined time value greater than the historical average pumping session time, such as, for example, between 2 minutes and 20 minutes greater than the historical average pumping session time, such as between about 2 minutes and about 15 minutes greater, such as between about 2 minutes and about 10 minutes greater, such as between about 2.5 minutes and about 7.5 minutes greater.

The notification element 208 can take any suitable form that allows for the controller to inform a user that one or more components of the breast pump 200 require maintenance or replacement. In the illustrated example, the notification element 208 is an indicator disposed on the housing 220 of the vacuum and control portion 203. The indicator can be, for example, a light source. However, it should be understood that the notification element 208 can take any other suitable form, such as, for example, an audible alert, other visual alerts displayed on a housing 220, an alert sent to an external device (e.g., a phone, a tablet, a computer), or any combination thereof. The visual alert can include an alert on the display screen 250, such as a symbol, text, backlighting on the display screen (e.g., a red background light or other atypical color), or the like. In some instances, the one or more buttons 248 can be configured to have backlighting (e.g., a red background light or other atypical color) that notifies the user. In examples that include sending an alert to an external device, the controller can be configured to wirelessly transmit the notification alert to the external device (e.g., via Bluetooth, Wi-Fi, etc.). For example, the alert can be sent to the external device by a software application notification, an email, a text message, or by any other suitable means. In these examples, the notification could also include a link to replacement component(s) that allows for a user to easily purchase such component(s).

The controller can be configured to inform a user that a specific component needs maintenance or replacement, or the controller can be configured to send a general alert to the user without specifying a specific component. For example, the notification element 208 can include multiple indicators with each component of the breast pump that may require maintenance or replacement having an indicator associated therewith. In some examples, text on a display can indicate specific component(s) that require maintenance or replacement, or an alert to an external device can specify which component(s) require maintenance or replacement. The one or more components that require maintenance or replacement can be components of the breast engagement portion 202 (e.g., tubing 224, the diaphragm assembly 210, the flange 212, the one-way valve 214, and/or the container 216) or other components of the breast pump 200 (e.g., the vacuum source).

In some instances, the breast pump 200 can include one or more sensors (not shown) that are configured to send signals to the controller to cause the controller to activate the notification element 208 and inform a user that one or more components of the breast pump 200 require maintenance or replacement. The sensor(s) can be positioned at any desired location on the breast pump and be configured to send a signal to the controller when the sensor(s) detect information that can be used by the controller to determine that one or more components require maintenance or replacement. The sensor(s) can include, for example, flow sensors, pressure sensors, proximity sensors, a sensor capable of detecting volume of liquid in a container, or any combination of these sensors. However, sensors can be costly and cause the breast pump 200 to be unnecessarily complex. Accordingly, while some implementations can include sensors, it should be understood that various implementations of the breast pumps are configured such that the controller 106 can determine whether one or more components of the breast pump require maintenance or replacement without obtaining information from sensor(s) to do so.

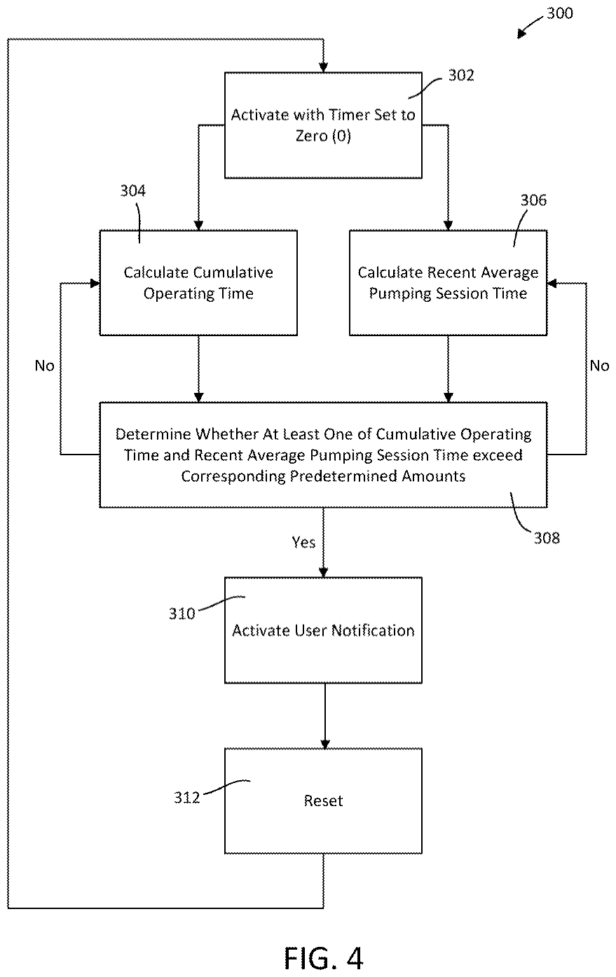

FIG. 4 illustrates an example method 300 performed by a controller of a breast pump. The breast pump and controller can take any suitable form, such as, for example, any form described in the present application. As shown at box 302, the method 300 includes the controller activating the breast pump with a timer set to zero (0). The controller can be configured to activate the breast pump based on a user input. As shown at box 304, method 300 further includes the controller calculating a cumulative operating time from the time that the breast pump was started (e.g., when the vacuum source was activated) with the timer set to zero (0). In other words, the method 300 includes the controller calculating the total time that the breast pump was active after the start of the method 300 at box 302. As shown at box 306, the method 300 includes the controller calculating a recent average pumping session time. The recent average pumping session time can be, for example, the average time for the previous two most recent pumping sessions, three most recent pumping sessions, four most recent pumping sessions, five more recent pumping sessions, etc.

As shown at box 308, the method 300 includes the controller determining whether at least one of the calculated cumulative operating time and the calculated recent average pumping session time exceeds a corresponding predetermined value. For example, the predetermined value for the cumulative operating time can be based on the amount of time one or more of the components are likely to perform sufficiently before wear and tear causes the component to decrease performance of the breast pump. The controller can be configured to compare the actual cumulative operating time to this predetermined value to determine if the actual cumulative operating time exceeds this value. The predetermined value for comparison with the recent average pumping session time can be based on a historical average pumping session time for the user. For example, the controller can be configured to determine a statistically significant variation of the historical average pumping session time and compare the recent average pumping session time to this statistically significant variation of the historical average pumping session time. The historical average pumping session time can be the average pumping session for every pumping session by the user less the number of recent pumping session times used to calculate the recent average pumping session time, or the historical average pumping session time can be the average time from predetermined pumping sessions. The statistically significant variation of the historical average pumping session time can be a predetermined percentage greater than the historical average pumping session time, or the statistically significant variation of the historical average pumping session time can be a predetermined time value greater than the historical average pumping session time.

When the controller determines that the calculated cumulative operating time exceeds the corresponding predetermined value and/or determines that the calculated recent average pumping session time exceeds the corresponding predetermined value, the method 300 moves to box 310 and the controller activates a user notification. If the controller determines that both the calculated cumulative operating time does not exceed the corresponding predetermined value and the calculated recent average pumping session time does not exceed the corresponding predetermined value, the method 300 returns to boxes 304, 306 and the controller continues to calculate the cumulative operating time and recent average pumping session time until at least one of these calculated times exceeds the corresponding predetermined value.

As shown at box 310, after the controller determines that at least one of the calculated cumulative operating time and recent average pumping session time exceeds the corresponding predetermined value, the method 300 includes the controller activating a user notification. The user notification can include, for example, an audible alert, a visual alert displayed on a housing (not shown) of the pump assembly, an alert sent to an external device (e.g., a phone, a tablet, a computer), or any combination thereof. The controller can be configured to inform a user that a specific component needs maintenance or replacement, or the controller can be configured to send a general alert to the user without specifying a specific component.

As shown at box 312, the method 300 further includes the controller resetting the timer to zero (0), as shown at box 302, after the maintenance is performed on the one or more components or the one or more components are replaced. In some instances, the controller is configured reset the breast pump based on a user input that the one or more components have undergone maintenance or replacement. In some instances, the breast pump can include one or more sensors that are able to detect if the one or more components have undergone maintenance or replacement and send a signal to the controller that causes the controller to reset the breast pump.

While the method 300 includes calculating both cumulative operating time and the recent average pumping session time and comparing these calculated times to corresponding predetermined values, it should be understood that the method 300 can include calculating only one of these times and comparing that calculated time to the corresponding predetermined value to determine if one or more components of the breast pump require maintenance or replacement. Also, it should be understood that the method 300 can include calculating any other time relating to the use of the breast pump that allows for a controller to determine whether one or more components of the breast pump require maintenance or replacement.

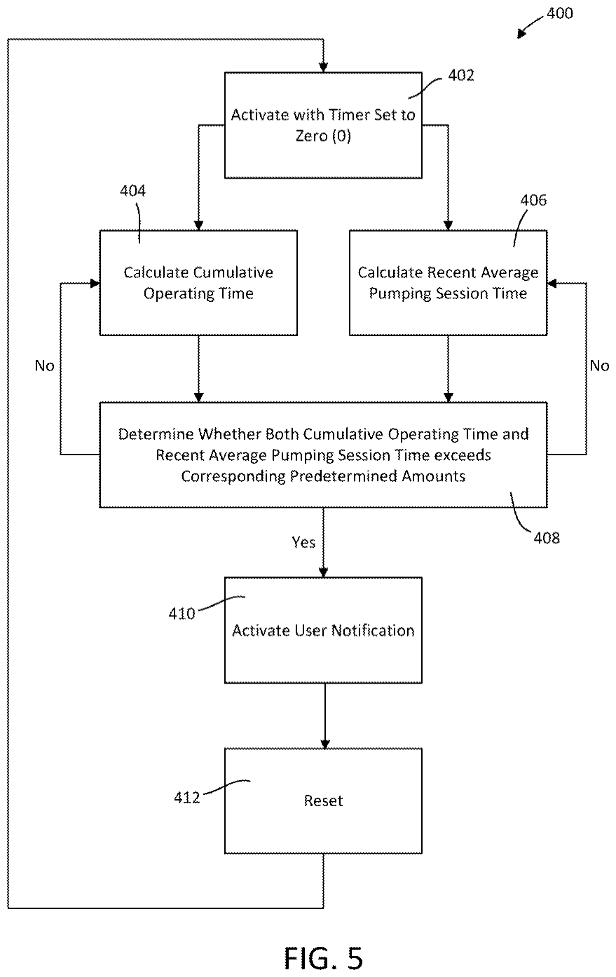

FIG. 5 illustrates an example method 400 performed by a controller of a breast pump. The breast pump and controller can take any suitable form, such as, for example, any form described in the present application. As shown at box 402, the method 400 includes the controller activating the breast pump with a timer set to zero (0). The controller can be configured to activate the breast pump based on a user input. As shown at box 404, method 400 further includes the controller calculating a cumulative operating time from the time that the breast pump was started (e.g., when the vacuum source was activated) with the timer set to zero (0). In other words, the method 400 includes the controller calculating the total time that the breast pump was active after the start of the method 400 at box 402. As shown at box 406, the method 400 includes the controller calculating a recent average pumping session time. The recent average pumping session time can be, for example, the average time for the previous two most recent pumping sessions, three most recent pumping sessions, four most recent pumping sessions, five more recent pumping sessions, etc.

As shown at box 408, the method 400 includes the controller determining whether both the calculated cumulative operating time and the calculated recent average pumping session time exceed corresponding predetermined values. For example, the predetermined value for the cumulative operating time can be based on the amount of time one or more of the components are likely to perform sufficiently before wear and tear causes the component to decrease performance of the breast pump. The controller can be configured to compare the actual cumulative operating time to this predetermined value to determine if the actual cumulative operating time exceeds this value. The predetermined value for comparison with the recent average pumping session time can be based on a historical average pumping session time for the user. For example, the controller can be configured to determine a statistically significant variation of the historical average pumping session time and compare the recent average pumping session time to this statistically significant variation of the historical average pumping session time. The historical average pumping session time can be the average pumping session for every pumping session by the user less the number of recent pumping session times used to calculate the recent average pumping session time, or the historical average pumping session time can be the average time from predetermined pumping sessions. The statistically significant variation of the historical average pumping session time can be a predetermined percentage greater than the historical average pumping session time, or the statistically significant variation of the historical average pumping session time can be a predetermined time value greater than the historical average pumping session time.

When the controller determines that both the calculated cumulative operating time and the calculated recent average pumping session time exceed the corresponding predetermined values, the method 400 moves to box 410 and the controller activates a user notification. If at least one of the cumulative operating time and the calculated recent average pumping session time do not exceed the corresponding predetermined value, the method 400 returns to boxes 404, 406 and the controller continues to calculate the cumulative operating time and recent average pumping session time until both of these calculated times exceed the corresponding predetermined value.

As shown at box 410, after the controller determines that both the calculated cumulative operating time and calculated recent average pumping session time exceeds the corresponding predetermined value, the method 400 includes the controller activating a user notification. The user notification can include, for example, an audible alert, a visual alert displayed on a housing (not shown) of the pump assembly, an alert sent to an external device (e.g., a phone, a tablet, a computer), or any combination thereof. The controller can be configured to inform a user that a specific component needs maintenance or replacement, or the controller can be configured to send a general alert to the user without specifying a specific component.

As shown at box 412, the method 400 further includes the controller resetting the timer to zero (0), as shown at box 402, after the maintenance is performed on the one or more components or the one or more components are replaced. In some instances, the controller is configured reset the breast pump based on a user input that the one or more components have undergone maintenance or replacement. In some instances, the breast pump can include one or more sensors that are able to detect if the one or more components have undergone maintenance or replacement and send a signal to the controller that causes the controller to reset the breast pump.

While the method 400 includes calculating both cumulative operating time and the recent average pumping session time and comparing these calculated times to corresponding predetermined values, it should be understood that the method 400 can include calculating any other time relating to the use of the breast pump that allows for a controller to determine whether one or more components of the breast pump require maintenance or replacement.

While various inventive aspects, concepts and features of the inventions may be described and illustrated herein as embodied in combination with exemplary embodiments, these various aspects, concepts and features may be used in many alternative embodiments, either individually or in various combinations and sub-combinations thereof. Unless expressly excluded herein, all such combinations and sub-combinations are intended to be within the scope of the present inventions. Still further, while various alternative embodiments as to the various aspects, concepts and features of the inventions--such as alternative materials, structures, configurations, methods, devices and components, alternatives as to form, fit and function, and so on—-may be described herein, such descriptions are not intended to be a complete or exhaustive list of available alternative embodiments, whether presently known or later developed. Those skilled in the art may readily adopt one or more of the inventive aspects, concepts or features into additional embodiments and uses within the scope of the present inventions even if such embodiments are not expressly disclosed herein.

Additionally, even though some features, concepts or aspects of the inventions may be described herein as being a preferred arrangement or method, such description is not intended to suggest that such feature is required or necessary unless expressly so stated. Moreover, while various aspects, features and concepts may be expressly identified herein as being inventive or forming part of an invention, such identification is not intended to be exclusive, but rather there may be inventive aspects, concepts and features that are fully described herein without being expressly identified as such or as part of a specific invention, Descriptions of exemplary methods or processes are not limited to inclusion of all steps as being required in all cases, nor is the order that the steps are presented to be construed as required or necessary unless expressly so stated.

1. A breast pump, comprising:
a vacuum source;
a breast engagement portion in fluid communication with the vacuum source, wherein the breast engagement portion is configured to engage a breast of a user to express breast milk; and
a controller in electrical communication with the vacuum source;
wherein the controller is configured to determine when the vacuum source is in an activated configuration and calculate a historical average pumping session time for the user, a statistically significant variation of the historical average pumping session time, and a recent average pumping session time for the user based on the determination that the vacuum source is in the activated configuration; and
wherein the controller is configured to notify a user that one or more components of the breast engagement portion require maintenance or replacement when the recent average pumping session time is greater than the statistically significant variation of the historical average pumping session time.

2. The breast pump according to claim 1, wherein the controller notifies the user via a user interface of the breast pump.

3. The breast pump according to claim 1, wherein the controller notifies the user via at least one of a light source of the breast pump, an audible alert, and an external device.

4. The breast pump according to claim 1, wherein the controller is configured to calculate a cumulative operating time that breast pump is in use based on the determination that the vacuum source is in the activated configuration, wherein the controller is configured to determine whether the cumulative operating time is greater than a predetermined value, and wherein the controller notifies the user that the one or more components of the breast engagement portion require maintenance or replacement when the cumulative operating time is greater than the predetermined value.

5. The breast pump according to claim 1, wherein the historical average pumping session time is calculated by calculating an average of pumping session times for predetermined pumping sessions.

6. The breast pump according to claim 1, wherein the statistically significant variation of the historical average pumping session time is between about 2% and about 50% greater than the historical average pumping session time.

7. The breast pump according to claim 1, wherein the recent average pumping session time is calculated by calculating an average time of at least three of the most recent pumping sessions.

8. The breast pump according to claim 1, wherein the breast engagement portion comprises:
a protection member;
a flange in fluid communication with the protection member;
a container in fluid communication with the flange; and
a one-way valve positioned between the flange and the container.

9. The breast pump according to claim 8, wherein the controller is configured to notify the user that at least one of the protection member and the one-way valve require maintenance or replacement.

10. The breast pump according to claim 9, wherein the protection member comprises a diaphragm assembly.

11. The breast pump according to claim 9, wherein the one-way valve comprises a duckbill valve.

12. A breast pump, comprising:
a vacuum source;
a breast engagement portion in fluid communication with the vacuum source, wherein the breast engagement portion is configured to engage a breast of a user to express breast milk; and
a controller in electrical communication with the vacuum source;
wherein the controller is configured to determine when the vacuum source is in an activated configuration and calculate a cumulative operating time of the breast pump based on the determination that the vacuum source is in the activated configuration; and
wherein the controller is configured to notify a user that one or more components of the breast engagement portion require maintenance or replacement when the cumulative operating time exceeds a predetermined value.

13. The breast pump according to claim 12, wherein the controller notifies the user via at least one of a user interface of the breast pump, a light source of the breast pump, an external device, and an audible alert.

14. The breast pump according to claim 12, wherein the breast engagement portion comprises:
a protection member;
a flange in fluid communication with the protection member;
a container in fluid communication with the flange; and
a one-way valve positioned between the flange and the container.

15. The breast pump according to claim 12, wherein the controller is configured to notify the user that at least one of the protection member and the one-way valve require maintenance or replacement.

16. A controller for a breast pump, the controller configured to perform the steps of:
calculating a cumulative operating time of the breast pump;
calculating a recent average pumping session time for a user;
calculating a historical average pumping session time for a user;
calculating a statistically significant variation of the historical average pumping session time;
determining at least one of:
the cumulative operating time exceeds a predetermined cumulative operating time value; and
the recent average pumping session time exceeds the statistically significant variation of the historical average pumping session time; and
activating a notification element to inform a user that one or more components of the breast pump require maintenance or replacement when the controller determines that at least one of the cumulative operating time exceeds the predetermined cumulative operating time value and the recent average pumping session time exceeds the statistically significant variation of the historical average pumping session time.

17. The breast pump according to claim 16, wherein the controller is configured to calculate the historical average pumping session time by calculating an average of pumping session times for predetermined pumping sessions.

18. The breast pump according to claim 16, wherein the controller is configured to calculate the statistically significant variation of the historical average pumping session time by calculating between about 2% and about 50% increase of the historical average pumping session time.

19. The breast pump according to claim 16, wherein the controller is configured to calculate the recent average pumping session time by calculating an average time of at least three of the most recent pumping sessions.

20. The breast pump according to claim 16, wherein the notification element includes at least one of a visual alert and an audible alert.
